(19) United States
(12) Patent Application Publication
(10) Pub. No.: US 2015/0057705 A1
(43) Pub. Date: Feb. 26, 2015

(54) Pursestring Epicardial Pad Device

(71) Applicant: Tendyne Holdings, Inc., Roseville, MN (US)

(72) Inventor: Robert M. Vidlund, Forest Lake, MN (US)

(73) Assignee: Tendyne Holdings, Inc., Roseville, MN (US)

(21) Appl. No.: 14/224,764

(22) Filed: Mar. 25, 2014

Related U.S. Application Data
(60) Provisional application No. 61/861,356, filed on Aug. 1, 2013.

(51) Int. Cl. A61B 17/04; A61B 17/00

(52) U.S. Cl. CPC A61B 17/0401; A61B 17/0057; A61B 2017/0414; A61B 2017/00659
USPC 606/228; 606/232

(57) ABSTRACT

This invention relates to a disk device for anchoring one or more transluminal sutures, and methods for anchoring such sutures, especially for anchoring transcatheter heart valves.

### CROSS REFERENCE TO RELATED APPLICATIONS

Not applicable.

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

none.

### BACKGROUND

1. Field of the Invention

The present invention relates generally to apparatus and methods for engaging tissue and/or closing openings through tissue, and more particularly to devices fix closing a puncture in the heart or other body lumen formed during a diagnostic or therapeutic procedure, and to methods for making and using such devices.

2. Background of the Invention

Catheterization and interventional procedures, such as angioplasty or stenting, generally are performed by inserting a hollow needle through a patient's skin and intervening tissue into the vascular system. A guide wire may then be passed through the needle lumen into the patient's blood vessel accessed by the needle. The needle may be removed, and an introducer sheath may be advanced over the guide wire into the vessel, e.g., in conjunction with or subsequent to a dilator. A catheter or other device may then be advanced through a lumen of the introducer sheath and over the guide wire into a position for performing a medical procedure. Thus, the introducer sheath may facilitate introducing various devices into the vessel, while minimizing trauma to the vessel wall and/or minimizing blood loss during a procedure.

Upon completing the procedure, the devices and introducer sheath may be removed, leaving a puncture site in the vessel wall. External pressure may be applied to the puncture site until clotting and wound sealing occur. This procedure, however, may be time consuming and expensive, requiring as much as an hour of a physician's or nurse's time. It is also uncomfortable for the patient, and requires that the patient remain immobilized in the operating room, catheter lab, or holding area. In addition, a risk of hematoma exists from bleeding before hemostasis occurs.

Various apparatus have been suggested for percutaneously sealing a vascular puncture by occluding the puncture site. For example, U.S. Pat. Nos. 5,192,302 and 5,222,974, issued to Kensey et al., describe the use of a biodegradable plug that may be delivered through an introducer sheath into a puncture site. When deployed, the plug may seal the vessel and provide hemostasis. Such devices, however, may be difficult to position properly with respect to the vessel, which may be particularly significant since it is generally undesirable to expose the plug material, e.g., collagen, within the bloodstream, Where it may float downstream and risk causing an embolism.

Another technique has been suggested that involves percutaneously suturing the puncture site, such as that disclosed in U.S. Pat. No. 5,304,184, issued to Hathaway et al. Percutaneous suturing devices, however, may require significant skill by the user, and may be mechanically complex and expensive to manufacture.

U.S. Pat. No. 5,478,354, issued to Tovey et al., discloses a surgical fastener including an annular base having legs that, in a relaxed state, extend in a direction substantially perpendicular to a plane defined by the base and slightly inwards toward one another. During use, the fastener is fit around the outside of a cannula, thereby deflecting the legs outward. The cannula is placed in an incision, and the fastener is slid along the cannula until the legs pierce into skin tissue. When the cannula is withdrawn, the legs move towards one another back to the relaxed state to close the incision.

U.S. Pat. Nos. 5,007,921 and 5,026,390, issued to Brown, disclose staples that may be used to close a wound or incision. In one embodiment, an “S” shaped staple is disclosed that includes barbs that may be engaged into tissue on either side of the wound. In another embodiment, a ring-shaped staple is disclosed that includes barbs that project from the ring. Sides of the ring may be squeezed to separate the barbs further, and the barbs may be engaged into tissue on either side of a wound. The sides may then be released, causing the barbs to return closer together, and thereby pulling the tissue closed over the wound. These staples, however, have a large cross-sectional profile and therefore may not be easy to deliver through a percutaneous site to close an opening in a vessel wall.

When the opening is made directly into the ventricular wall or apex of the heart, such as when a prosthetic valve is percutaneously delivered, and deployed, the efficacy of sealing the puncture site is critical to the life of the patient since hemodynamic losses from a cardiac puncture will cause shock and death within minutes. Further, the outward pressure that the puncture site is subjected to, when it is located in the heart muscle itself, is much higher than puncture sites that are distal to the heart. Accordingly, devices for engaging and closing tissue, e.g., to close a cardiac puncture site, would be considered useful to solve these and other problems known in the art.

### BRIEF SUMMARY OF THE INVENTION

What is provided herein is a device for anchoring a transluminal suture, composing a substantially rigid suturing disk having an axial tunnel, a locking pin tunnel that intersects the axial tunnel, a locking pin operatively associated with the locking pin tunnel, one or more radial channels that do not intersect with the axial tunnel and that do not intersect the locking pin tunnel, and a winding channel circumferentially disposed within a perimeter sidewall of the disk.

In preferred embodiments, the device further comprises a polyester velour coating.

In preferred embodiments, the device further comprises wherein the one or more radial channels is four radial channels.

In preferred embodiments, the device further comprises wherein the one or more radial channels each have a proximal end comprising an enlarged axial keyhole tunnel and a distal end comprising an opening in the disk edge.

In preferred embodiments, the device further comprises a flexible pad operatively associated with the rigid suturing disk, said flexible pad having a through-hole longitudinally aligned with the axial tunnel of the suturing disk.

In preferred embodiments, the device further comprises a sleeve gasket operatively associated with the rigid suturing disk, said sleeve gasket having a lumen longitudinally aligned with the axial tunnel of the suturing disk.

In preferred embodiments, the device further comprises a sleeve gasket attached to the rigid suturing disk and a flexible pad attached to the sleeve gasket, wherein the sleeve gasket has a lumen longitudinally aligned with said axial tunnel of the suturing disk, and wherein the flexible pad has a through-hole longitudinally aligned with both the lumen of the sleeve gasket and the axial tunnel of the suturing disk.

In preferred embodiments, there is also provided a device for anchoring a transluminal suture, comprising a substantially rigid suturing disk, a sleeve gasket connected to the suturing disk, and a flexible pad connected to the sleeve gasket, said substantially rigid suturing disk having an axial tunnel, a locking pin tunnel that intersects the axial tunnel, a locking pin operatively associated with the locking pin tunnel, one or more radial channels that do not intersect with the axial tunnel and that do not intersect the locking pin tunnel, and a winding channel circumferentially disposed within a perimeter sidewall of the disk, said sleeve gasket in longitudinal alignment with said axial tunnel, said flexible pad having a through-hole longitudinally aligned with both the lumen of the sleeve gasket and the axial tunnel of the suturing disk.

In preferred embodiments, the invention also includes a method for anchoring a transluminal suture, comprising the step of affixing the transluminal suture to the device described herein, wherein the device is positioned external to a body lumen, and the transluminal suture extends from within the lumen to the device.

In preferred embodiments, the invention also includes the device in a sterile surgical kit, the device in a sterile surgical kit containing a transcatheter delivery system, and the device in a sterile surgical kit containing a transcatheter valve.

### BRIEF DESCRIPTION OF THE DRAWINGS

FIG. 1 is a side view of one embodiment of the present invention. FIG. 1 shows a flexible pad for contact with the epicardial surface, a sleeve gasket, and a rigid suturing disk.

FIG. 2 is a side view of one embodiment of the present invention with a suturing tether in the context of a ventricular wall. FIG. 2 shows the intraventricular suturing tether extending through the flexible pad, sleeve gasket and rigid suturing disk prior to applying the device to the puncture site and making contact with the epicardial surface.

FIG. 3 is a side view of one embodiment of the present invention with a suturing tether in the context of a ventricular wall being slid into its final position. FIG. 3 shows the intraventricular suturing tether extending through the flexible pad, sleeve gasket and rigid suturing disk, and the device being applied to the puncture site and making contact with the epicardial surface.

FIG. 4 is an exploded perspective view of one embodiment of the present invention and shows the flexible pad, sleeve gasket and a rigid suturing disk with locking pin.

FIG. 5 is an exploded perspective view of one embodiment of the present invention without the sleeve gasket, and shows the flexible pad and a rigid suturing disk with locking pin.

FIG. 6 is an exploded perspective view of one embodiment of the present invention without the flexible pad, and shows the sleeve gasket and a rigid suturing disk with locking pin.

FIG. 7 is a perspective view of the distal side of one embodiment of the present inventive suturing disk.

FIG. 8 is a perspective view of the proximal side of one embodiment of the present inventive suturing disk.

### DETAILED DESCRIPTION OF THE INVENTION

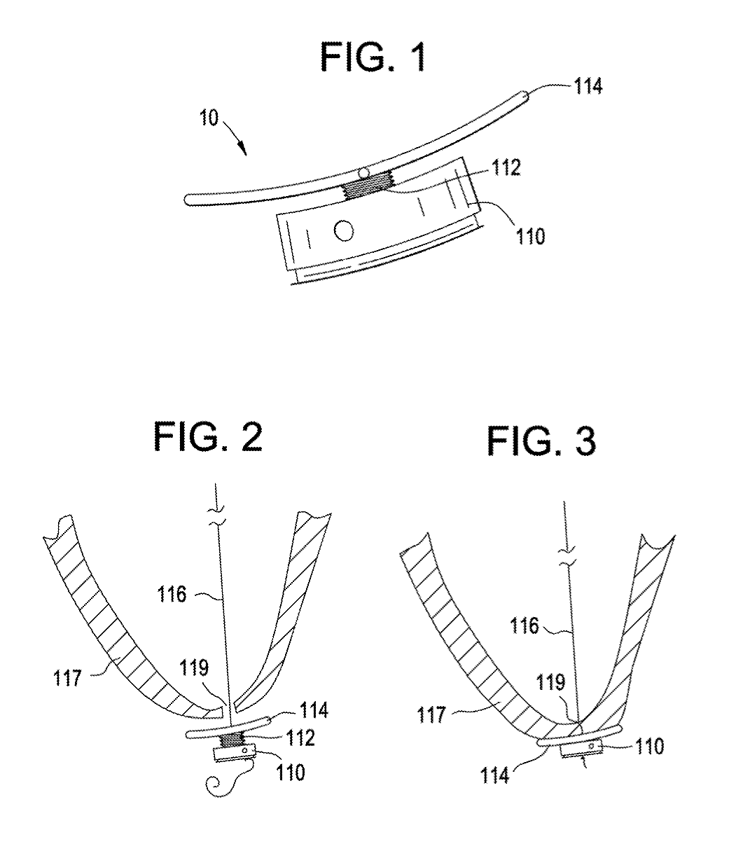

Referring now to the drawings, FIG. 1 is a side view of one embodiment of the present invention 10 and shows a flexible pad 114 for contact with the epicardial surface, a sleeve gasket 112, and a rigid suturing disk 110. The flexible pad 114 is intended for contacting the epicardial surface and may be constructed of any suitable biocompatible surgical material. The pad 114 functions to assist sealing of the surgical puncture. In a preferred embodiment, it is made having a double-layered velour coating to promote ingrowth of the pad 114 into the puncture site area. Pads, or felt pledgets, are commonly made of a felted polyester and may be cut to any suitable size or shape, such as those available from Bard® as PTFE Felt Pledgets having a nominal thickness of 2.87 mm. In a preferred embodiment, the pad 114 is larger in diameter than the suturing disk 110.

The sleeve gasket 112 functions to seal any gap or leakage that may occur between the pad 114 and the suturing disk 110. The sleeve gasket 112 is made of a flexible material so that it can be compressed when the disk 110 and/or pad 114 are tightened against the puncture site (e.g. against the ventricular wall). The sleeve gasket 112 may be connected to the pad 114 and the disk 110 as an integral assemblage, or the components may be separately slid onto the suturing tether, in order, and then tightened against the puncture site (e.g., in the ventricular wall). The sleeve gasket 112 functions to prevent hemodynamic leakage that may flow along the path of the axially located suturing tether. Such anchoring tethers are used in deployment of prosthetic. heart valves and need to extend from within the lumen of the organ being anchored (e.g., the heart), to the external anchoring location (e.g., the epicardial surface). It is also contemplated that the present invention may be used to anchor one or more suturing tethers in other surgical situations where such tether(s) is required to extend from an intraluminal cavity to an external anchoring site.

The rigid suturing disk 110 functions to provide the anchoring and mounting platform to which one or more suturing tethers may be tied. The disk 110 may be made of any suitable biocompatible material. In a preferred embodiment, it is made of polyethylene, or other hard or semi-hard polymer, and is covered with a polyester velour to promote ingrowth. In other embodiments, it is made of metal such as Nitinol®, or ceramic materials. The disk range in size depending on the particular need. In a preferred embodiment, it ranges from 1.0-3.0 cm in diameter. In other embodiments, it ranges from 0.2-5.0 cm, the larger size not necessarily for intraventricular anchoring but for other surgical use (e.g., hernia repair), gastrointestinal repairs, etc.

One benefit of using a disk to capture and anchor a suture is that, unlike suture anchors that bore into tissue with screws or barbs, there is little or no trauma to the tissue at the site of the anchor. Further, using a disk, which quickly slides over the tether, instead of stitches, allows for the effective permanent closure of large punctures. Surgically closing large punctures by sewing takes time and is difficult. When closing a puncture in the heart, adding the difficulty of requiring a surgeon to sew the puncture closed increases the likelihood of life threatening complications to the patient. This is especially so in situations where a prosthetic heart valve is delivered and deployed without opening the chest cavity using transcatheter technologies. Sewing a ventricular puncture closed in this situation is not tenable.

FIG. 2 is a side view of one embodiment of the present invention with a suturing tether 116 in the context of a ventricular wall 117. FIG. 2 shows the intraventricular suturing tether 116 extending through the ventricular puncture 119, flexible pad 114, sleeve gasket 112, and rigid suturing disk 110. FIG. 2 shows the device before it is tightened down onto the epicardial surface, and just prior to applying the device to the puncture site. FIG. 2 also shows how tether 116 may be wound around disk 110 to improve anchoring. FIG. 2 shows sleeve gasket 112 in an uncompressed state.

FIG. 3 is a side view of one embodiment of the present invention with a suturing tether 116 in the context of a ventricular wall 117 being slid into its final position and shows the intra ventricular suturing tether 116 extending through the flexible pad 114, sleeve gasket (not shown), and rigid suturing disk 110, and the device being applied to the puncture site 119 and making contact with the epicardial surface. FIG. 3 shows how tether 116 may be trimmed after it is affixed to the disk 110.

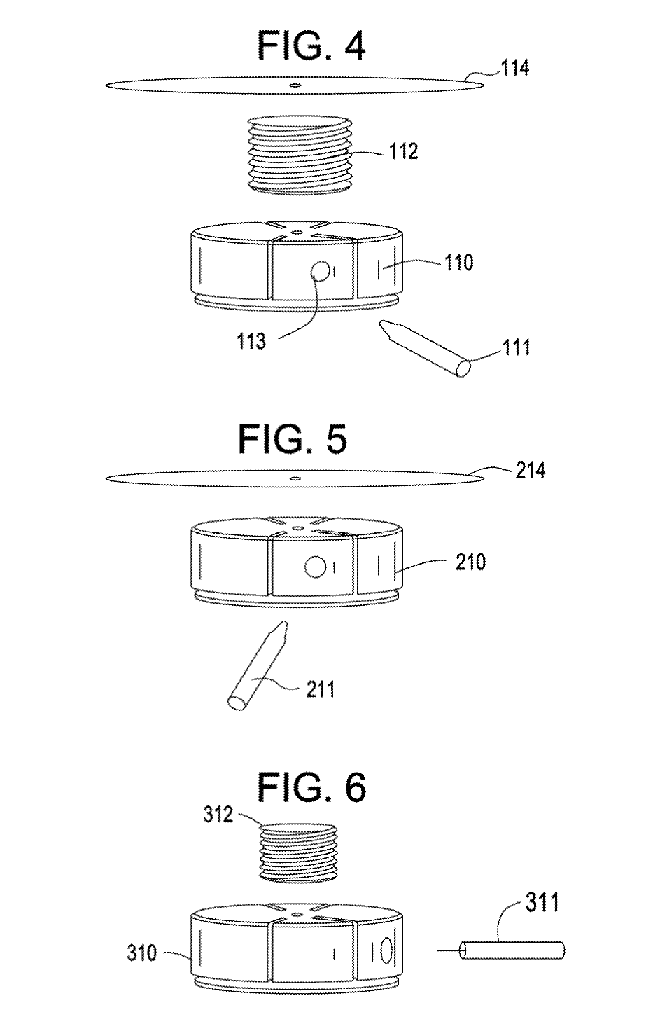

FIG. 4 is an exploded perspective view of one embodiment of the present invention and shows the flexible pad 114, sleeve gasket 112, and to rigid suturing disk 110 with locking pin 111. The locking pin 111 functions to hold the suturing tether (not shown) in place after the disk 110 is tightened against the ventricular wall by piercing the suturing tether as it travels axially through the disk 110. The locking pin hole 113 on disk 110 allows the locking pin 111 to laterally intersect and affix the longitudinally disposed suturing tether.

FIG. 5 is an exploded perspective view of one embodiment of the present invention without the sleeve gasket, and shows the flexible pad 214, and a rigid suturing disk 210 with locking pin 211. This embodiment is used where an anti-leakage sleeve is unnecessary.

FIG. 6 is an exploded perspective view of one embodiment of the present invention without the flexible pad, and shows the sleeve gasket 312, and a rigid suturing disk 310 with locking pin 311. This embodiment is used where a flexible pad is unnecessary.

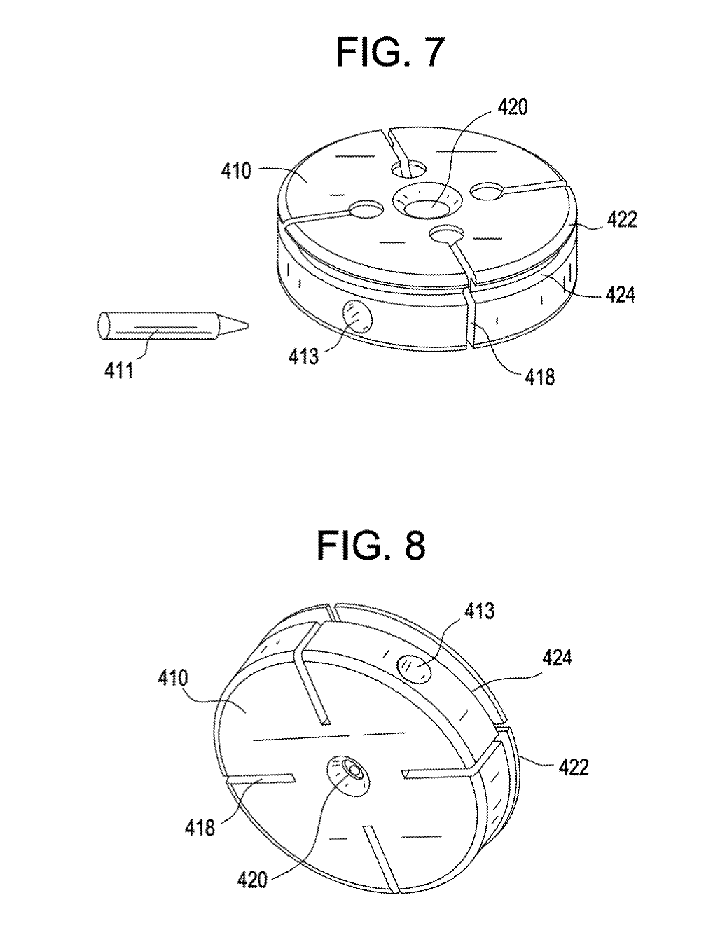

FIG. 7 is a perspective view of the distal side of one embodiment of the present inventive suturing disk 410. FIG. 7 shows locking pin 411 and locking pin hole 413, which act in concert to laterally intersect and affix the suturing tether (not shown) as it axially travels through axial tunnel or aperture 420. Axial tunnel 420 may, in preferred embodiments, be tapered to allow the suture to he easily threaded into the axial tunnel and to reduce lateral cutting force of the disk 410 against the suture. Radial channel 41$ functions to allow a user to quickly capture and seat a suturing tether (not shown) that is intended to be anchored. Flange 422 defines winding channel 424 and allows a user to quickly wind suture tether(s) around disk 410. Using the winding channel 424 in conjunction with the radial channel(s) 418 allows a user to quickly anchor the suture, while permitting the user to unwind and recalibrate so that the tether tension is appropriate for the particular situation. In a preferred embodiment, a suture that anchors a transcatheter valve will have about 2 lbs. of longitudinal force.

FIG. 8 is a perspective view of the proximal side of one embodiment of the present inventive suturing disk 410. FIG. 8 shows locking pin hole 413 which acts in concert with a locking pin (not shown) to laterally intersect and affix the suturing tether (not shown) as it axially travels through axial tunnel 420. Axial tunnel 420 may, in preferred embodiments, be tapered to allow the suture to be easily threaded into the axial tunnel and to reduce lateral cutting force of the disk 410 against the suture. Radial channel 418 functions to allow a user to quickly capture and seat a suture tether not shown) that is intended to be anchored. Flange 422 defines winding, channel 424 and allows a user to quickly wind suture tether(s) around disk 410. Using the winding channel 424 in conjunction with the radial channel(s) 418 allows a user to quickly anchor the suture, while permitting the user to unwind and recalibrate the tether tension.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may he altered or that insubstantial changes may be made without departing from the scope of the invention. Accordingly, the scope of the invention is determined by the scope of the following claims and their equitable Equivalents.

What is claimed is:

1. A device for anchoring a transluminal suture, comprising a substantially rigid suturing disk having an axial tunnel, a locking pin tunnel that intersects the axial tunnel, a locking pin operatively associated with the locking pin tunnel, one or more radial channels that do not intersect with the axial tunnel and that do not intersect the locking pin tunnel, and a winding channel circumferentially disposed within a perimeter sidewall of the disk.

2. The device of claim 1, further comprising a polyester velour coating.

3. The device of claim 1, wherein the one or more radial channels is four radial channels.

4. The device of claim 1, further comprising wherein the one or more radial channels, each have a proximal end comprising an enlarged axial keyhole tunnel and a distal end comprising an opening in the disk edge.

5. The device of claim 1, further comprising a flexible pad operatively associated with the rigid suturing disk, said flexible pad having a through-hole longitudinally aligned with said axial tunnel of the suturing disk.

6. The device of claim 1, further comprising a sleeve gasket operatively associated with the rigid suturing disk, said sleeve gasket having a lumen longitudinally aligned with said axial tunnel of the suturing disk.

7. The device of claim 1, further comprising a sleeve gasket attached to the rigid suturing disk and a flexible pad attached to the sleeve gasket, wherein the sleeve gasket has a lumen longitudinally aligned with said axial tunnel of the suturing disk, and wherein the flexible pad has a through-hole longitudinally aligned with both the lumen of the sleeve gasket and the axial tunnel of the suturing disk.

8. The device of claim 1 in a sterile surgical kit.

9. The device of claim 1 in a sterile surgical kit containing a transcatheter delivery system.

10. The device of claim 1 in a sterile surgical kit containing a transcatheter valve.

11. A device for anchoring a transluminal suture, comprising a substantially rigid suturing disk, a sleeve gasket connected to the suturing disk, and a flexible pad connected to the sleeve gasket, said substantially rigid suturing disk having an axial tunnel, a locking pin tunnel that intersects the axial tunnel, a locking pin operatively associated with the locking pin tunnel, one or more radial channels that do not intersect with the axial tunnel and that do not intersect the locking pin tunnel, and a winding channel circumferentially disposed within a perimeter sidewall of the disk, said sleeve gasket in longitudinal alignment with said axial tunnel of the suturing disk, said flexible pad having a through-hole longitudinally aligned with both the lumen of the sleeve gasket and the axial tunnel of the suturing disk.

12. A method for anchoring a transluminal suture, comprising the step of affixing the transluminal suture to the device of claim 1, wherein the device is positioned external to a body lumen, and the transluminal suture extends from within the lumen to the device.
